(19) United States
(12) Patent Application Publication
(10) Pub. No.: US 2018/0104914 A1
(43) Pub. Date: Apr. 19, 2018

(54) POROUS STRUCTURE WITH IMPROVED POROSITY, METHOD FOR PRODUCING THE POROUS STRUCTURE, POROUS HIERARCHICAL STRUCTURE AND METHOD FOR PRODUCING THE POROUS HIERARCHICAL STRUCTURE

(71) Applicants: IUCF-HYU (INDUSTRY-UNIVERSITY COOPERATION FOUNDATION HANYANG UNIVERSITY), Seoul (KR); INTELLIGENT SYNTHETIC BIOLOGY CENTER, Daejeon (KR)

(72) Inventors: Dong Rip KIM, Seoul (KR); Min Soo JEON, Seoul (KR); Jeong Hun HWANG, Seoul (KR); Sun Chang KIM, Daejeon (KR); Byung Kwan CHO, Daejeon (KR); Chang Sung HEU, Seoul (KR)

(73) Assignees: IUCF-HYU (INDUSTRY-UNIVERSITY COOPERATION FOUNDATION HANYANG UNIVERSITY), Seoul (KR); INTELLIGENT SYNTHETIC BIOLOGY CENTER, Daejeon (KR)

(21) Appl. No.: 15/572,370

(22) PCT Filed: May 4, 2016
(86) PCT No.: PCT/KR2016/004742

§ 371 Date: Nov. 7, 2017

(30) Foreign Application Priority Data
May 7, 2015 (KR) 10-2015-0063962
May 7, 2015 (KR) 10-2015-0063963

(51) Int. Cl. B29C 67/20; C12M 1/16; C12P 1/04; C25B 1/04; B29C 33/38; C01B 32/05; C04B 35/515; C04B 38/06

(52) U.S. Cl. CPC B29C 67/202; C12M 1/16; C12P 1/04; C25B 1/04; B29D 99/0089; C01B 32/05; C04B 35/515; C04B 38/067; B29C 33/3814

(57) ABSTRACT

A porous structure according to one embodiment of the present invention is constituted by a frame having a plurality of pores interconnected 3-dimensionally through a plurality of connecting passages. The plurality of pores defined by the frame are distributed in a closest packed state and are interconnected 3-dimensionally through a plurality of connecting passages in a symmetric structure, thus being effective in achieving a maximum porosity of the porous structure. A porous hierarchical structure according to one embodiment of the present invention includes a first porous structure having a plurality of 3-dimensionally interconnected first pores and a second porous structure having a plurality of 3-dimensionally interconnected second pores whose diameter is different from that of the first pores and surrounding and bonded to the first porous structure. A porous hierarchical structure according to a further embodiment of the present invention includes a frame having a plurality of 3-dimensionally interconnected first pores having a diameter in the micrometer range and a plurality of 3-dimensionally interconnected second pores formed around the first pores and whose diameter is smaller than that of the first pores.

### TECHNICAL FIELD

The present invention relates to a porous structure and a method for producing the same, and more particularly to a porous structure in which the area of connecting passages between pores is increased, achieving improved porosity, and a method for producing the porous structure. The present invention also relates to a porous hierarchical structure and a method for producing the same, and more particularly to a porous hierarchical structure in which internal pores and external pores have different pore sizes and a method for producing the porous hierarchical structure.

### BACKGROUND ART

In recent years, the reserves of fossil fuels as major causes of environmental pollution have been depleted. Under such circumstances, active efforts have been made to replace fossil fuels. For example, new concepts of microbial chemical production systems as well as microbial fuel cells have received attention as approaches to produce electricity or fuel based on the use of microbes.

Particularly, considerable research efforts have recently concentrated on the supply of electricity or hydrogen produced from sunlight, which has attracted attention as an inexhaustible source of clean energy, to microbes where electro-biosynthesis for chemical production is induced by the supplied electricity or hydrogen.

A success of this research and development can provide substitutes for the production of both energy sources and chemicals for which the petrochemical industry is currently responsible and is expected to greatly contribute to the reduction of carbon dioxide emissions.

The development of structures capable of maximizing microbial electro-biosynthesis for chemical production is of importance comparable to the development of microbes for mass production of high value-added chemicals. Such structures are required to be compatible with microbes, have large specific surface area, and be highly electrically conductive in order to maximize the supply of electricity or hydrogen to microbes. The former two requirements are associated with the attachment of the largest possible number of microbes.

Particularly, 3-dimensional porous structures are needed that have the largest possible specific surface area taking into consideration that microbes have a size of several to several tens of micrometers.

In this connection, several research results were reported. For example, Zhang et al. (2013) found that the production of acetate from Sporomusa ovata attached to a chitosan-coated carbon cloth electrode as a biocathode is 6-7 times larger than when attached to a general carbon cloth electrode (see Non-Patent Document 1). The reason was reported to be because the density of cells attached to the chitosan-coated electrode is higher by 9 times than that that of cells attached to the general carbon cloth electrode.

However, such porous structures suffer from difficulties in controlling the pore size, trapping microbes larger than the pores, and fundamentally preventing microbes from escaping.
Patent Document 1: Korean Patent Publication No. 10-2013-0021150
(Non-Patent Document 1) Zhang, T. et al. (2013) “Improved cathode materials for microbial electrosynthesis”. Energy Environ. Sci. 6, 217.

### DETAILED DESCRIPTION OF THE INVENTION

### Problems to be Solved by the Invention

The present invention has been made in view of the above problems, and it is one object of the present invention to provide a porous structure in which the area of connecting passages between pores is increased, achieving improved porosity.

It is a further object of the present invention to provide a method for producing a porous structure with improved porosity in which the area of connecting passages between pores is increased.

It is another object of the present invention to provide a porous hierarchical structure in which internal pores and external pores have different pore sizes so that microbes of various sizes can be trapped and fundamentally prevented from escaping.

It is still another object of the present invention to provide a method for producing a porous hierarchical structure in which internal pores and external pores have different pore sizes so that microbes of various sizes can be trapped and fundamentally prevented from escaping.

### Means for Solving the Problems

A porous structure according to one embodiment of the present invention is constituted by a frame having a plurality of pores interconnected 3-dimensionally through a plurality of connecting passages.

The frame is made of a material selected from carbon materials, metal materials, and metal oxides.

The pores have a diameter in the micrometer range.

Four connecting passages extend downwardly, four connecting passages extend laterally, and four connecting passages extend upwardly from the central pore.

A method for producing a porous structure according to a further embodiment of the present invention includes (A) constructing and stacking a plurality of sacrificial templates, (B) pressurizing and heating the stack of the sacrificial templates, (C) adding a gel precursor to the pressurized and heated stack of the sacrificial templates, followed by gelation, and (D) carbonizing the stack of the sacrificial templates including the gelled gel precursor.

Step (A) includes (A-1) preparing a plurality of sacrificial templates in the form of spheres by a polymerization reaction using a polymer or an oxide and (A-2) drying the plurality of sacrificial templates during slow cooling or freezing to stack the plurality of sacrificial templates.

Optionally, the plurality of sacrificial templates may be arranged in a tetragonal packed array by dielectrophoresis in an ethanolic solution.

Additionally, the sacrificial templates may be stacked by floating the sacrificial templates (e.g., polystyrene sacrificial templates) using a solution having a higher density (e.g., ethylene glycol) and evaporating the solution to array and arrange the sacrificial templates.

In sub-step (A-2), the stack includes a hexagonal closest packed structure and a cubic closest packed structure.

Sub-step (A-2) is carried out at a temperature lower than 15° C.

Step (B) is carried out at a temperature different by ±50° C. from the glass transition temperature of the material for the sacrificial templates.

Step (B) is carried out at a pressure of 10 to 500 kPa and a temperature of 110 to 150° C.

In step (C), the gel precursor includes resorcinol, formaldehyde, sodium carbonate, and deionized (DI) water. The gel precursor may further use toluenesulfonic acid and calcium carbonate as additives. The use of the additives improves the strength and hardness of a final porous structure.

In step (C), the gel precursor is added under a reduced pressure of 0.1 atm.

In step (D), the carbonization is performed in a nitrogen atmosphere at a temperature of 800-1000° C. for 2-3 hours.

A porous hierarchical structure according to one embodiment of the present invention includes a first porous structure having a plurality of 3-dimensionally interconnected first pores and a second porous structure having a plurality of 3-dimensionally interconnected second pores whose diameter is different from that of the first pores and surrounding and bonded to the first porous structure.

The porous hierarchical structure further includes an electrode disposed on one outer surface of the second porous structure.

Each of the first porous structure and the second porous structure of the porous hierarchical structure is made of a material selected from carbon materials, metal materials, and metal oxides.

The first pores have a diameter in the micrometer range and the second pores have a diameter smaller than that of the first pores.

The porous hierarchical structure further includes a third porous structure having a plurality of third pores whose diameter is smaller than that of the second pores and surrounding the second porous structure and a fourth porous structure having a plurality of fourth pores whose diameter is smaller than that of the third pores and surrounding the third porous structure.

A method for producing a porous hierarchical structure according to one embodiment of the present invention includes (A) constructing a first porous structure having a plurality of 3-dimensionally interconnected first pores and (B) constructing a second porous structure having a plurality of 3-dimensionally interconnected second pores whose diameter is different from that of the first pores and surrounding and bonded to the first porous structure.

The method further includes (C) constructing a third porous structure having a plurality of third pores whose diameter is smaller than that of the second pores and surrounding the second porous structure and (D) constructing a fourth porous structure having a plurality of fourth pores whose diameter is smaller than that of the third pores and surrounding the third porous structure.

Step (A) includes (A-1) constructing primary sacrificial templates whose size corresponds to that of first pores, (A-2) adding a primary gel precursor to a stack of the primary sacrificial templates, followed by gelation, and (A-3) primarily carbonizing the gelled stack of the primary sacrificial templates to form a first porous structure. The preparation of the first porous structure in step (A) is not limited. For example, the first porous structure may be replaced by a commercial porous carbon structure having pores whose size is on the order of tens to hundreds of micrometers.

Step (B) includes (B-1) introducing a filler into the first porous structure, (B-2) constructing a plurality of secondary sacrificial templates whose size corresponds to that of second pores, (B-3) drying the filler-containing first porous structure and applying and stacking the plurality of dried filler-containing secondary sacrificial templates to and on the outer surface of the first porous structure, (B-4) adding a secondary gel precursor to the stack of the secondary sacrificial templates, followed by gelation, and (B-5) secondarily carbonizing the stack of the secondary sacrificial templates including the gelled secondary gel precursor to form a second porous structure.

Sub-step (A-1) includes (A-11) preparing a plurality of primary sacrificial templates in the form of spheres using a polymer or an oxide, (A-12) drying the plurality of primary sacrificial templates to stack the plurality of primary sacrificial templates, and (A-13) pressurizing and heating the stack of the primary sacrificial templates.

In step (A-2), the primary gel precursor is prepared by mixing resorcinol, formaldehyde, sodium carbonate, and deionized (DI) water. The primary gel precursor may further use toluenesulfonic acid and calcium carbonate as additives. The use of the additives improves the strength and hardness of a final porous hierarchical structure.

The material for the filler is the same as that for the primary sacrificial templates.

The method further includes plating one surface of the second porous structure to form an electrode.

A porous hierarchical structure according to a further embodiment of the present invention includes a frame having a plurality of 3-dimensionally interconnected first pores having a diameter in the micrometer range and a plurality of 3-dimensionally interconnected second pores formed around the first pores and whose diameter is smaller than that of the first pores.

The frame is made of a material selected from carbon materials, metal materials, and metal oxides.

The porous hierarchical structure further includes a plurality of 3-dimensionally interconnected third pores formed around the first pores and whose diameter is smaller than that of the second pores.

The porous hierarchical structure further includes a plurality of 3-dimensionally interconnected fourth pores formed around the first pores and whose diameter is smaller than that of the third pores.

A method for producing a porous hierarchical structure according to another embodiment of the present invention includes (A) constructing sacrificial templates having different sizes corresponding to micrometer- to nanometer-sized pores by a polymerization reaction, (B) mixing the sacrificial templates in a predetermined mass ratio and drying the sacrificial templates to stack the sacrificial templates, (C) pressurizing and heating the mixed sacrificial template stack, (D) adding a primary gel precursor to the mixed sacrificial template stack, followed by gelation, and (E) primarily carbonizing the mixed sacrificial template stack including the gelled primary gel precursor.

Step (A) includes (A-1) preparing a plurality of sacrificial templates in the form of spheres using a polymer or an oxide, (A-2) adding the sacrificial templates to a solution containing a surfactant and a swelling agent and primarily stirring the solution, (A-3) mixing the primary stirred solution with a cross-linking agent, a polymerization initiator, and a monomer and secondarily stirring the solution, and (A-4) polymerizing the secondary stirred solution under heat.

The sacrificial templates having various sizes can be constructed by varying the composition of the solution in step (A) and repeating step (A). Alternatively, a mixture of sacrificial templates having various sizes may be used in step (A).

In step (B), the sacrificial templates constructed by polymerization are mixed with an ethanolic solution, dried, and stacked.

In step (C), the pressure and temperature conditions are adjusted depending on the desired contact area between the sacrificial templates.

Step (C) is carried out at a pressure of 10 to 500 kPa and a temperature of 110 to 150° C.

In step (D), the primary gel precursor is prepared by mixing resorcinol, formaldehyde, sodium carbonate, and deionized (DI) water. The primary gel precursor may further use toluenesulfonic acid and calcium carbonate as additives. The use of the additives improves the strength and hardness of a final porous structure.

The amount of the monomer is determined depending on the desired sizes of the pores.

Features and advantages of the present invention will become more apparent from the detailed description set forth below with reference to the accompanying drawings.

It should be understood that the terms and words used in the specification and the claims are not to be construed as having common and dictionary meanings but are construed as having meanings and concepts corresponding to the technical spirit of the present invention in view of the principle that the inventor can define properly the concept of the terms and words in order to describe his/her invention with the best method.

### Effects of the Invention

The porous structure according to one embodiment of the present invention is constituted by a frame having a plurality of pores distributed in a closest packed state and interconnected 3-dimensionally through a plurality of connecting passages in a symmetric structure, achieving a maximum porosity.

The method for producing a porous structure according to a further embodiment of the present invention is effective in producing a porous structure that has a plurality of pores distributed in a closest packed state and interconnected 3-dimensionally through a plurality of connecting passages in a symmetric structure, achieving a maximum porosity of the porous structure.

The porous hierarchical structure according to an embodiment of the present invention is effective in preventing microbes from escaping.

The porous hierarchical structure according to an embodiment of the present invention allows the culture of microbes of various sizes in each pore. Therefore, the use of the porous hierarchical scaffold enables the production of various chemicals and is effective in producing new chemicals through reactions of chemicals produced from microbes cultured in each pore.

The method for producing a porous hierarchical structure according to an embodiment of the present invention facilitates the production of a porous hierarchical structure that has smaller diameter pores formed around larger diameter pores. Due to this construction, the porous hierarchical structure is suitable for preventing grown microbes from escaping and culturing microbes of various sizes in the pores.

### BRIEF DESCRIPTION OF THE DRAWINGS

FIG. 1 is a perspective diagram of a porous structure according to one embodiment of the present invention.

FIG. 2 is a flow chart explaining a method for producing a porous structure according to a further embodiment of the present invention.

FIGS. 3a to 3d are exemplary diagrams explaining a method for producing a porous structure according to a further embodiment of the present invention.

FIG. 4 is a SEM image of a porous structure produced in Comparative Example 1.

FIG. 5 is a SEM image of a porous structure produced in Comparative Example 2.

FIG. 6 is a SEM image of a porous structure produced in Comparative Example 3.

FIG. 7 is a SEM image of a porous structure produced in Example 1.

FIG. 8 is a SEM image of a porous structure produced in Example 2.

FIG. 9 shows exemplary diagrams explaining the production of a porous structure in Example 2.

FIG. 10 shows SEM images of (a) a porous structure produced in Comparative Example 4 and (b) a porous structure produced in Example 2.

FIG. 11 is a cross-sectional diagram of a porous hierarchical structure according to a first embodiment of the present invention.

FIG. 12 shows SEM images of (a) portion A, (b) portion B, and (c) portion C of FIG. 11.

FIG. 13 shows exemplary diagrams showing the trapping of microbes with a porous hierarchical structure according to a first embodiment of the present invention.

FIG. 14 is a flow chart explaining a method for producing a porous hierarchical structure according to a first embodiment of the present invention.

FIG. 15 shows exemplary diagrams explaining a method for producing a porous hierarchical structure according to a first embodiment of the present invention.

FIG. 16 is an exemplary diagram of a porous hierarchical structure according to a second embodiment of the present invention.

FIG. 17 shows SEM images of a platinum-plated porous hierarchical structure according to an embodiment of the present invention.

FIG. 18 shows cross-sectional diagrams of a porous hierarchical structure according to a third embodiment of the present invention.

FIG. 19 is an exemplary diagram showing a porous hierarchical structure according to a fourth embodiment of the present invention.

FIG. 20 is a flow chart explaining a method for producing a porous hierarchical structure according to a third embodiment of the present invention.

FIG. 21 shows exemplary diagrams explaining a method for producing a porous hierarchical structure according to a third embodiment of the present invention.

FIG. 22 shows SEM images of a porous hierarchical structure according to a third embodiment of the present invention.

### BEST MODE FOR CARRYING OUT THE INVENTION

Other objects, advantages, and novel features of the invention will become more apparent from the following detailed description and preferred embodiments with reference to the appended drawings. In the drawings, the same elements are denoted by the same reference numerals even though they are depicted in different drawings. Although the terms as “first” and “second,” etc. may be used to describe various elements, these elements should not be limited by above terms. These terms are used only to distinguish one element from another. In the description of the present invention, detailed explanations of related art are omitted when it is deemed that they may unnecessarily obscure the essence of the present invention.

Preferred embodiments of the present invention will be described in detail with reference to the accompanying drawings. FIG. 1 is a perspective diagram of a porous structure according to one embodiment of the present invention.

The porous structure 100 according to one embodiment of the present invention is constituted by a frame 110 having a plurality of pores A interconnected 3-dimensionally through a plurality of connecting passages C, as shown in FIG. 1.

Specifically, the frame 110 may be formed using a carbon material, a metal material, such as nickel (Ni), copper (Cu) or silicon, or a metal oxide, such as titanium dioxide (TiO₂). The diameter of the pores A may be in the micrometer range.

As shown in FIG. 1, the pores A have a diameter in the micrometer range and are interconnected 3-dimensionally through the plurality of connecting passages C. The plurality of connecting passages C are formed by face-to-face contact of a plurality of sacrificial templates 101, which will be explained below. The plurality of connecting passages C are portions where the plurality of sacrificial templates 101 are in face-to-face contact with each other to form a closest packed structure. The plurality of connecting passages C may form a symmetric structure in which four connecting passages extend downwardly, four connecting passages extend laterally, and four connecting passages extend upwardly from the central pore.

As another example, the plurality of pores are in the form of spheres, a plurality of unit structures, each of which is a body centered structure in which eight different pores are arranged adjacent to the central pore and are in communication with the central pore through the plurality of connecting passages, are arranged in a continuous array, and the plurality of connecting passages form a symmetric structure in which four connecting passages extend downwardly, four connecting passages extend laterally, and four connecting passages extend upwardly from the central pore.

The porosity of the porous structure 100 can be maximized because the plurality of pores A defined by the frame 110 having a minimum volume are distributed in a closest packed state and are interconnected 3-dimensionally through the plurality of connecting passages C in a symmetric structure.

### MODE FOR CARRYING OUT THE INVENTION

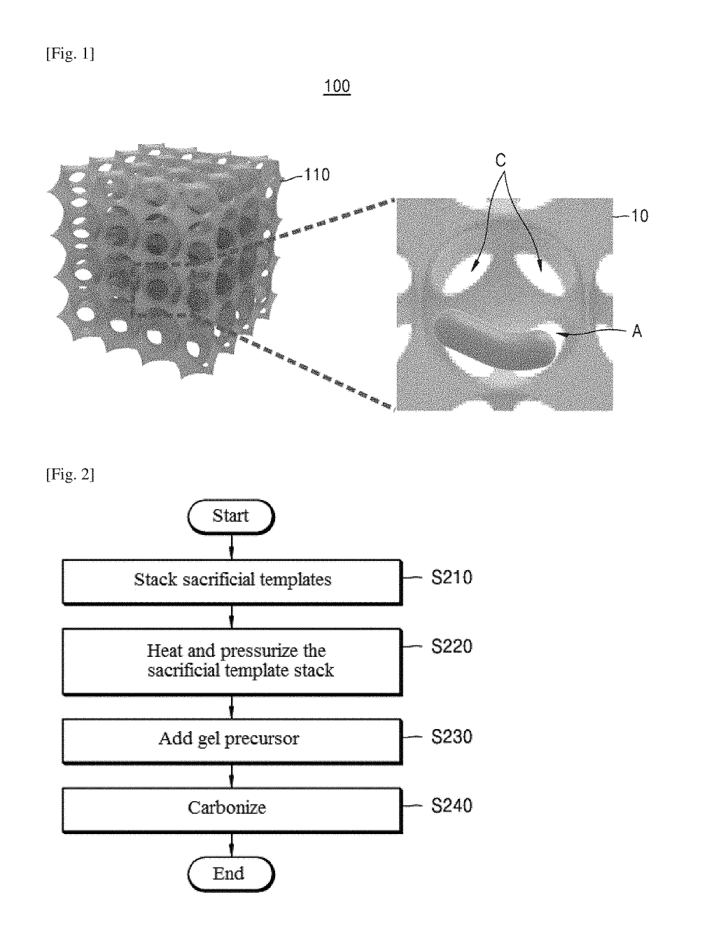

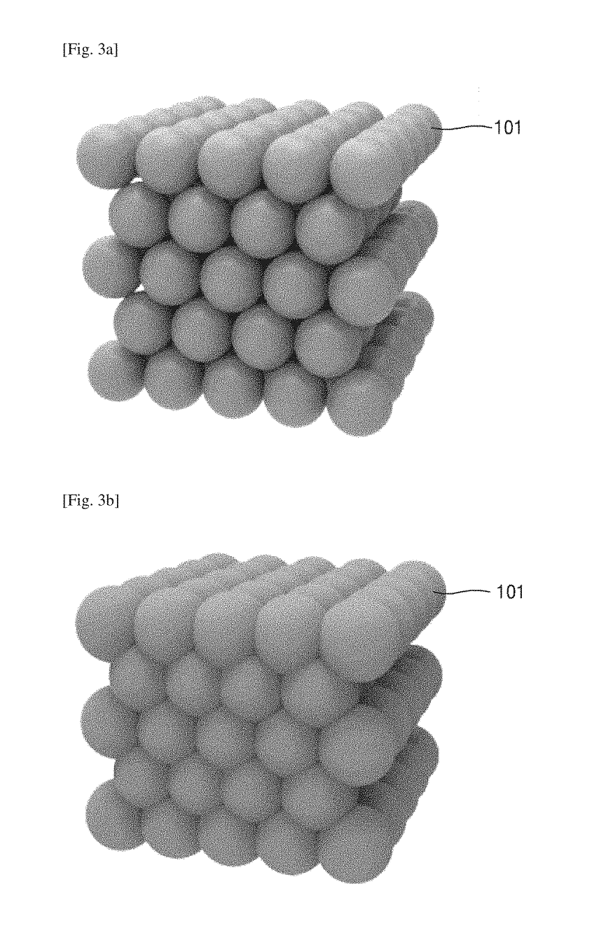

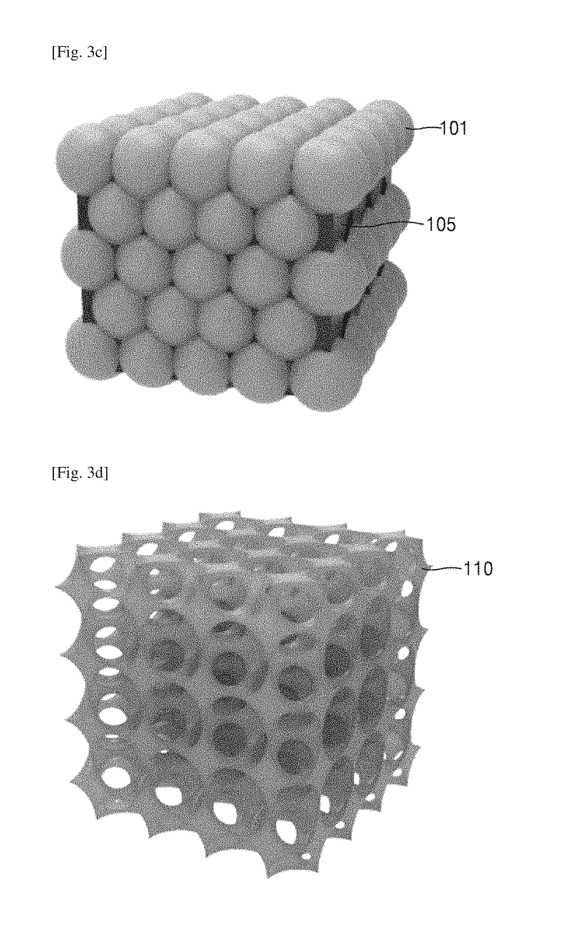

A method for producing a porous structure according to a further embodiment of the present invention will be explained with reference to FIGS. 2 and 3a to 3d. FIG. 2 and FIGS. 3a to 3d are a flow chart and exemplary diagrams explaining the method, respectively.

According to the method, a porous structure is produced by the following procedure. First, a plurality of sacrificial templates 101 having a size corresponding the size of pores A are constructed by a polymerization reaction and are stacked on one another (S210).

Specifically, the plurality of sacrificial templates 101 are in the form of spheres and can be constructed using a polymer, such as polystyrene (PS), polymethyl methacrylate (PMMA) or polypropylene (PP), or an oxide, such as silicon dioxide (SiO₂) or titanium dioxide (TiO₂), as shown in FIG. 3a.

Thereafter, the plurality of sacrificial templates are immersed in an ethanolic solution and slowly dried or freeze-dried at a temperature not higher than 15° C. As a result, the plurality of sacrificial templates 101 form a stack, which can be precipitated, as shown in FIG. 3a. Optionally, dielectrophoresis may be used to arrange the plurality of sacrificial templates 101 in a tetragonal packed structure in an ethanolic solution. Additionally, the sacrificial templates may be stacked by floating the sacrificial templates (e.g., polystyrene sacrificial templates) using a solution having a higher density (e.g., ethylene glycol) and evaporating the solution to array and arrange the sacrificial templates.

For example, the resulting stack may include a hexagonal closest packed structure or a cubic closest packed structure.

After the ethanolic solution is completely evaporated, the stack of the sacrificial templates 101 is pressurized and heated (S220). As a result, the contact area between the sacrificial templates 101 in the stack is enhanced.

The contact area between the sacrificial templates 101 is determined by 3-dimensional connecting passages C between the pores A. Thus, the pressure and temperature conditions can be determined depending on the material for the sacrificial templates 101 and the size of the contact area. The heating is performed at a temperature different by ±50° C. from the glass transition temperature of the material for the sacrificial templates 101.

For example, when the sacrificial templates 101 have a diameter in the micrometer range and are made of a polymeric material, they may be pressurized to 10-500 kPa and heated to 110-150° C.

As a result of the pressurization and heating (S220), the contact area between the sacrificial templates 101 is enhanced and the plurality of sacrificial templates 101 are densely packed in the stack.

A gel precursor 105 is added to the stack of the densely packed sacrificial templates 101, followed by gelation (S230).

For example, the gel precursor 105 may be prepared by stirring resorcinol, formaldehyde, sodium carbonate, and deionized (DI) water in a molar ratio of 50:100:1:300. The gel precursor may further use toluenesulfonic acid and calcium carbonate as additives. The use of the additives improves the strength and hardness of a final porous structure.

The gel precursor 105 is added to the closest packed stack of the sacrificial templates 101, as shown in FIG. 3c, followed by gelation by heating at 50-80° C. for 48-72 hours. Here, the gel precursor 105 is easily added under a reduced pressure of 0.1 atm or less.

The stack of the sacrificial templates 101 including the gelled gel precursor 105 is carbonized in a nitrogen atmosphere, for example, by heating at 800-1000° C. for 2-3 hours (S240).

By this carbonization, the sacrificial templates 101 are removed and the gelled gel precursor 105 is carbonized to form a frame 110, as shown in FIG. 3d. The frame 110 constitutes a porous structure 110 having a plurality of pores A interconnected 3-dimensionally through a plurality of connecting passages C.

Therefore, the method enables the production of a porous structure 100 with a maximum porosity in which a plurality of pores A are distributed in a closest packed state and are interconnected 3-dimensionally through a plurality of connecting passages C in a symmetric structure.

The efficiency of the method will be demonstrated with reference the following examples, including comparative examples.

### Comparative Example 1

The above procedure is repeated, except that the stack of the sacrificial templates 101 is not pressurized and heated.

### Comparative Example 2

The above procedure is repeated, except that the stack of the sacrificial templates 101 is not pressurized but is heated to 30° C.

### Comparative Example 3

The above procedure is repeated, except that the stack of the sacrificial templates 101 is not heated but is pressurized to 10 kPa.

### Comparative Example 4

The above procedure is repeated, except that the stack of the sacrificial templates 101 formed in S201 is dried at room temperature.

### Example 1

The above procedure is repeated, except that the stack of the sacrificial templates 101 is pressurized to 10 kPa and heated to 130° C. in S220.

### Example 2

The above procedure is repeated, except that the stack of the sacrificial templates 101 is formed by freeze-drying (S210) and is pressurized to 15 kPa and heated to 150° C. (S220).

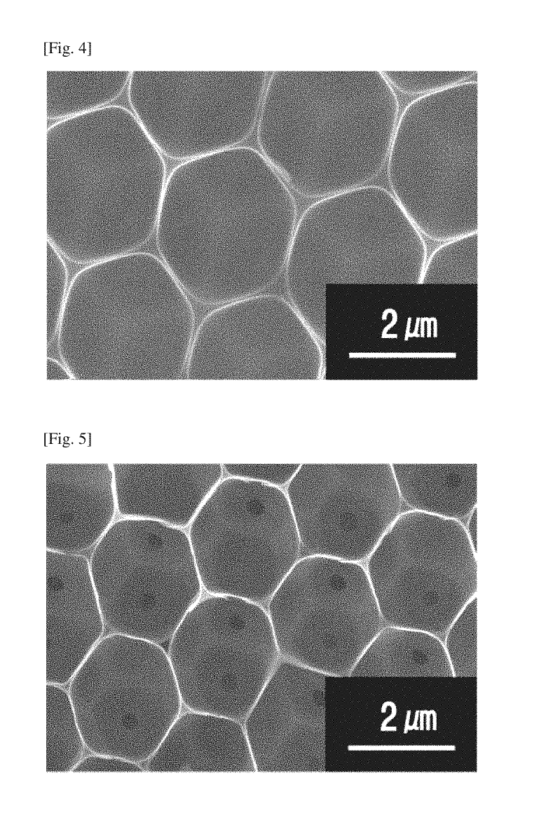

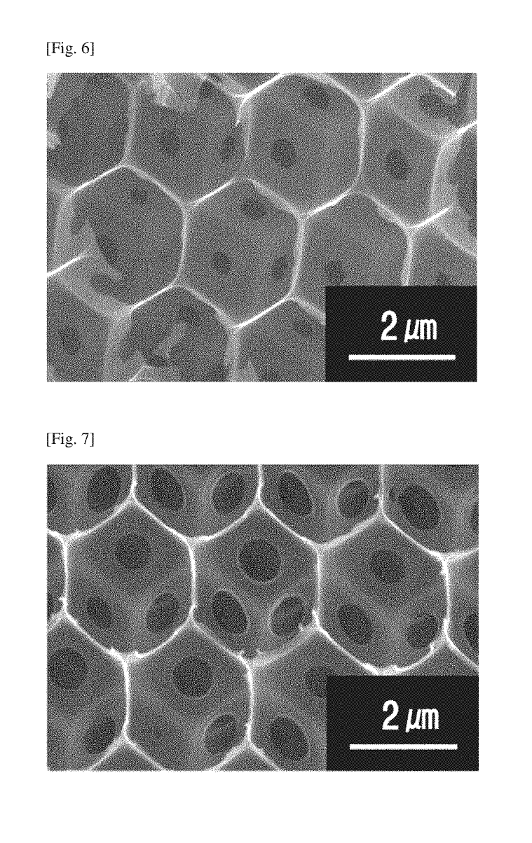

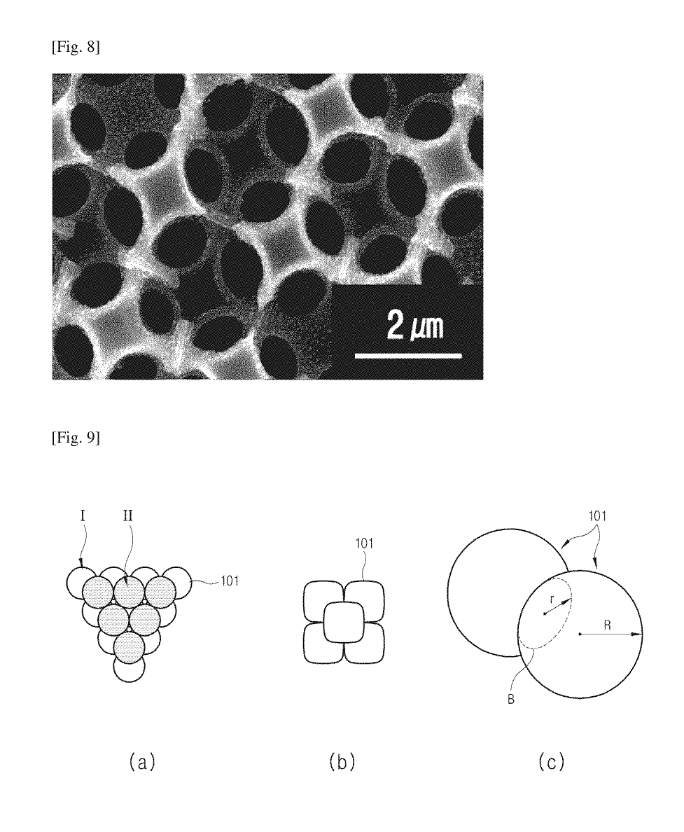

SEM images of the porous structures produced in Comparative Examples 1-4 and Examples 1-2 are shown in FIGS. 4 to 7 and 10. The porous structure produced in Comparative Example 1 has no connecting passages between pores (FIG. 4). Negligibly small connecting passages are formed in the porous structures of Comparative Examples 1 (FIG. 5) and 2 (FIG. 6).

The connecting passages between the plurality of pores in the porous structure of Comparative Example 4, which is produced by drying the stack of the sacrificial templates 101 at room temperature (S210), are irregular ((a) of FIG. 10) compared to the connection passage between the pores in the porous structure of Example 2 (FIG. 10b).

In contrast, the connecting passages between the plurality of pores in the porous structures of in Examples 1 and 2, which are produced by pressurizing and heating the stacks of the sacrificial templates 101 (S220), are formed regularly and expanded, as shown in FIGS. 7 and 8.

Particularly, FIG. 8 shows the porous structure produced by freeze-drying the stack of the sacrificial templates 101 in Example 2.

Specifically, the stack of the sacrificial templates 101 has a structure in which three sacrificial templates 101 overlap in the first layer I and one sacrificial template 101 is stacked thereon in the second layer III when freeze-dried, as shown in FIG. 9a, and the sacrificial templates 101 in the layers I and II are deformed and arrayed when pressurized to 15 kPa and heated to 150° C. (S220) so that one sacrificial template 101 in the second layer II is stacked on four sacrificial templates 101 in the first layer I to form a closest packed structure, as shown in FIG. 9b.

The closest packed structure of FIG. 9b has portions B where the sacrificial templates 101 are in face-to-face contact with each other, as shown in FIG. 9c. Particularly, the closest packed structure f FIG. 9b has a total of 12 portions B consisting of four lower portions B, four lateral portions B, and four upper portions B.

By the subsequent carbonization S240, the portions B form connecting passages C, as shown in FIG. 8 and (b) of FIG. 10.

Therefore, the porosity of the porous structure produced by the method can be improved because the frame 110 has a minimum volume by the closest packed structure and the pores A are interconnected 3-dimensionally through the plurality of connecting passages C.

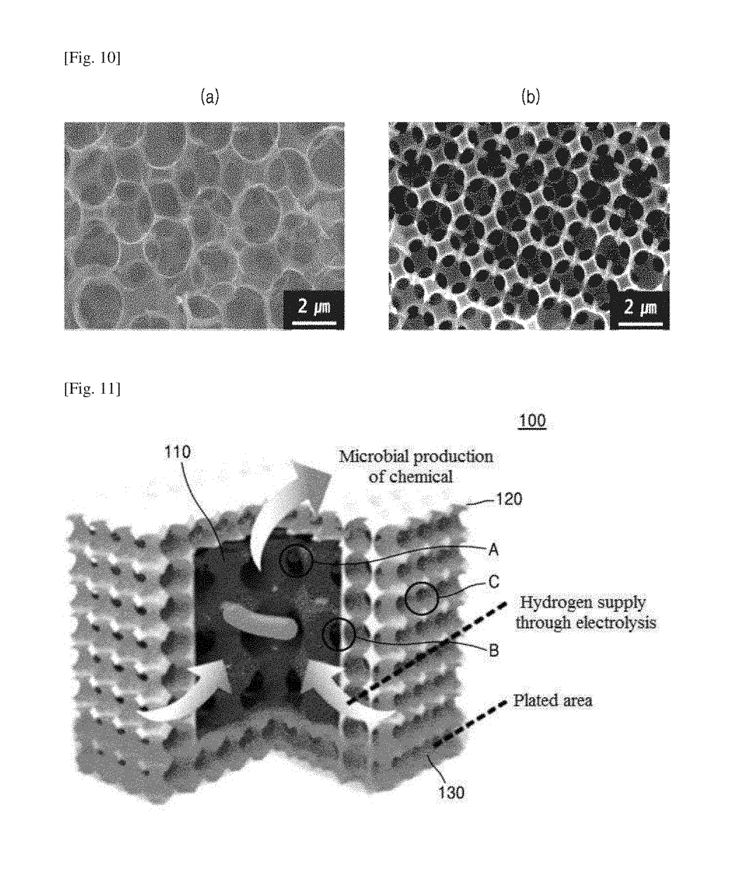

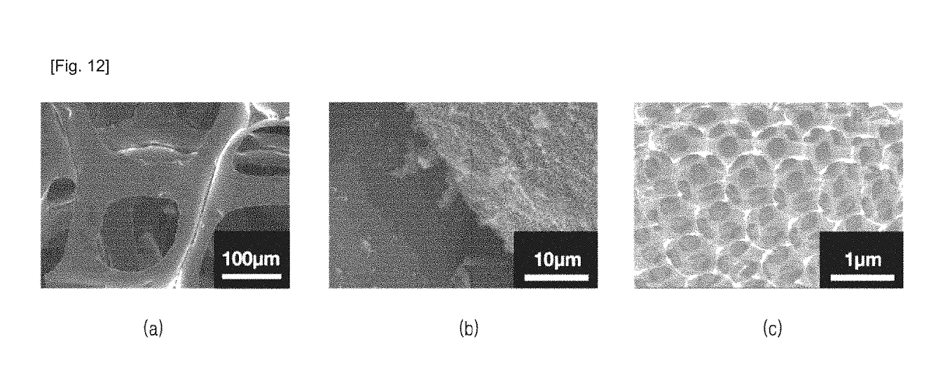

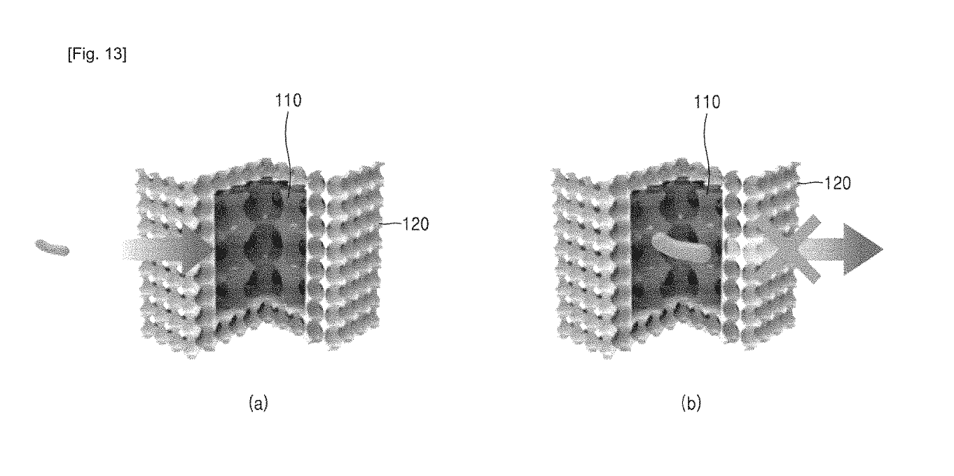

FIG. 11 is a cross-sectional diagram of a porous hierarchical structure according to a first embodiment of the present invention, FIG. 12 shows SEM images of (a) portion A, (b) portion B, and (c) portion C of FIG. 11, and FIG. 13 shows exemplary diagrams showing the trapping of microbes with the porous hierarchical structure.

The porous hierarchical structure 100 according to the first embodiment of the present invention may include a first porous structure 110 having 3-dimensionally interconnected first pores, a second porous structure 120 having a plurality of 3-dimensionally interconnected second pores whose diameter is different from that of the first pores and surrounding and bonded to the first porous structure 110, and optionally, an electrode 130 disposed on one outer surface of the second porous structure 120, as shown in FIG. 11.

Each of the first porous structure 110 and the second porous structure 120 may be constituted by a frame made of a carbon material, a metal material, such as nickel (Ni), copper (Cu) or silicon, or a metal oxide, such as titanium oxide (TiO₂). The first pores of the first porous structure 110 may have a diameter different from the second pores of the second porous structure 120. Particularly, the first pores of the first porous structure 110 have a diameter in the micrometer range, as shown in (a) of FIG. 12, and the second pores of the second porous structure 120 may have a diameter smaller than the first pores, as shown in (c) of FIG. 12. For example, the second pores of the second porous structure 120 may have a diameter in the nanometer range.

The electrode 130 may be optionally formed on one outer surface of the second porous structure 120, for example, by electroplating with a conductive metal, such as aluminum or platinum.

In the porous hierarchical structure 100, the inner first porous structure 110 having first pores is bonded to the outer second porous structure 120 having second pores whose diameter is smaller than that of the first pores. Due to this construction, microbes at the initial stage of growth can enter the inner first porous structure 110 through the outer second porous structure 120 having second pores, as shown in (a) of FIG. 13.

Thereafter, the microbes proliferate in the first porous structure 110, resulting in an increase in population, as shown in (b) of FIG. 13. Accordingly, the grown microbes fail to escape through the second pores of the second porous structure 120, considerably reducing the probability that the microbes will escape from the porous hierarchical structure 100.

The stability between the microbes and the porous structures 110 and 120 is satisfied by the carbon-made frames constituting the first porous structure 110 and the second porous structure 120. At the same time, hydrogen can be easily supplied by electrolysis using the electrode 130, which may be optionally plated with platinum nanoparticles. That is, the supply of electricity through the electrode 130 induces electrolysis of water in the electrode 130 to produce hydrogen. The produced hydrogen escapes from the porous hierarchical structure 110 due to its lighter weight than water. At this time, the microbes present in the first porous structure 110 can absorb hydrogen to produce chemicals.

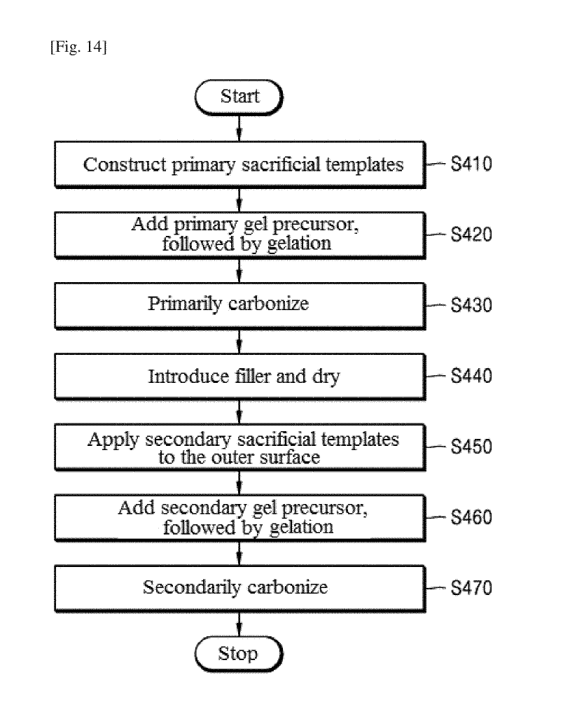

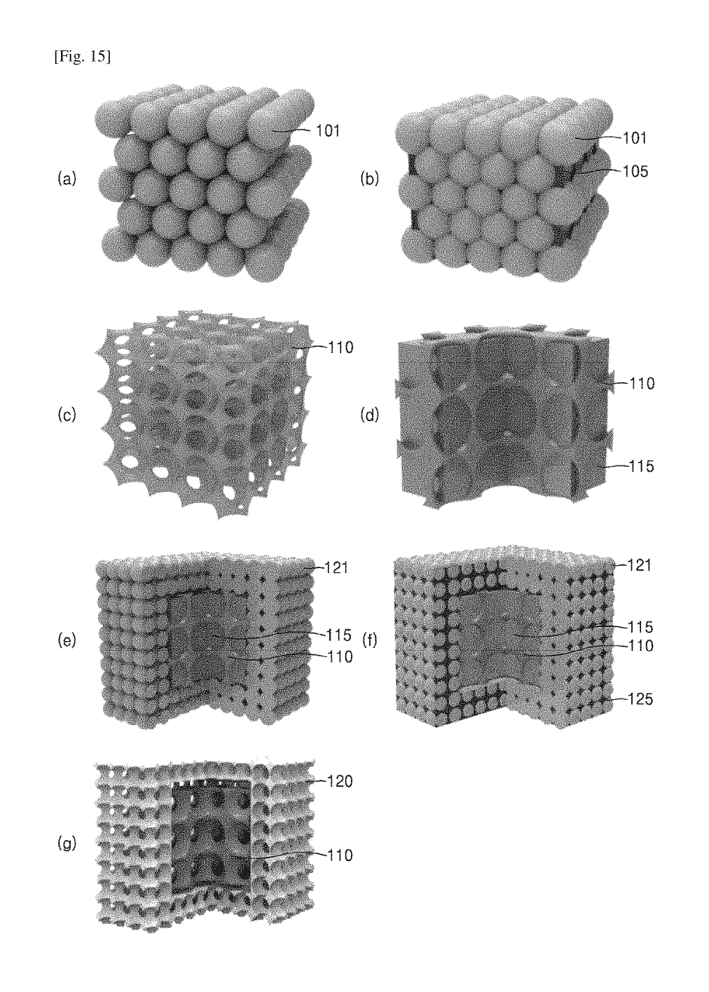

A method for producing the porous hierarchical structure according to the first embodiment of the present invention will be explained with reference to FIGS. 14 and 15a to 15g. FIGS. 14 and 15 are a flow chart and exemplary diagrams explaining the method.

According to the method, the porous hierarchical structure is produced by the following procedure. First, primary sacrificial templates 101 having a size corresponding to first pores are constructed by a polymerization reaction (S410), as shown in FIG. 14.

Specifically, the primary sacrificial templates 101 are in the form of spheres and can be constructed using a polymer, such as polystyrene (PS), polymethyl methacrylate (PMMA) or polypropylene (PP), or an oxide, such as silicon dioxide (SiO₂) or titanium dioxide (TiO₂), as shown in (a) of FIG. 15.

Thereafter, the plurality of primary sacrificial templates 101 are immersed in an ethanolic solution and dried at a refrigeration temperature not higher than 10° C. As a result, the plurality of primary sacrificial templates 101 form a stack, which can be precipitated. Optionally, dielectrophoresis may be used to arrange the plurality of sacrificial templates 101 in a tetragonal packed structure in an ethanolic solution.

Additionally, the sacrificial templates may be stacked by floating the sacrificial templates (e.g., polystyrene sacrificial templates) using a solution having a higher density (e.g., ethylene glycol) and evaporating the solution to array and arrange the sacrificial templates.

After the ethanolic solution is completely evaporated, the stack of the primary sacrificial templates 101 is pressurized to 10-500 kPa and heated to 110-150° C. As a result, the contact area between the primary sacrificial templates 101 in the stack is enhanced.

The contact area between the primary sacrificial templates 101 is determined by 3-dimensional interconnection structures between the first pores. Thus, the pressure and temperature conditions can be determined depending on the material for the primary sacrificial templates 101 and the size of the contact area.

Then, a primary gel precursor 105 is added to the stack of the primary sacrificial templates 101, followed by gelation by heating (S420).

For example, the primary gel precursor 105 may be prepared by stirring resorcinol, formaldehyde, sodium carbonate, and deionized (DI) water in a molar ratio of 50:100:1:300. The primary gel precursor may further use toluenesulfonic acid and calcium carbonate as additives. The use of the additives improves the strength and hardness of a porous structure.

The primary gel precursor 105 is added to the stack of the primary sacrificial templates 101, as shown in (b) of FIG. 15, followed by gelation by heating at 50-80° C. for 48-72 hours. Here, the primary gel precursor 105 is easily added under a reduced pressure of 0.1 atm or less.

The stack of the primary sacrificial templates 101 including the gelled primary gel precursor 105 is primarily carbonized in a nitrogen atmosphere, for example, by heating at 800-1000° C. for 2-3 hours (S430).

By this primary carbonization, the primary sacrificial templates 101 are removed and the gelled primary gel precursor 105 is carbonized to form a frame 110 made of a carbon material, as shown in (c) of FIG. 15. The frame 110 constitutes a first porous structure 100. The preparation of the first porous structure is not limited. For example, the first porous structure may be replaced by a commercial porous carbon structure having pores whose size is on the order of tens to hundreds of micrometers.

Thereafter, a filler 15 is introduced into the first porous structure 110, followed by drying (S440), as shown in (d) of FIG. 15.

The filler 115 may be prepared using the same material for the primary sacrificial templates 101, i.e. a polymer, such as polystyrene (PS), polymethyl methacrylate (PMMA) or polypropylene (PP), or an oxide, such as silicon dioxide (SiO₂) or titanium dioxide (TiO₂).

Subsequently, a plurality of secondary sacrificial templates 121 having a size corresponding to second pores are constructed by a polymerization reaction and are stacked on and applied to the outer surface of the dried first porous structure 110 containing the filler 115 (S450).

Specifically, the secondary sacrificial templates 121 have a size corresponding to second pores and may be prepared using the same material for the primary sacrificial templates 101, i.e. a polymer, such as polystyrene (PS), polymethyl methacrylate (PMMA) or polypropylene (PP), or an oxide, such as silicon dioxide (SiO₂) or titanium dioxide (TiO₂). For example, the secondary sacrificial templates 121 may be in the form of spheres.

Thereafter, the plurality of secondary sacrificial templates 121 are stacked on and applied to the outer surface of the dried first porous structure 110 containing the filler 115 and are heated to a temperature of 110 to 150° C., as shown in (e) of FIG. 15.

A secondary gel precursor 125 is added to the stack of the secondary sacrificial templates 121, followed by gelation by heating (S460).

Like the primary gel precursor 105, the secondary gel precursor 125 may be prepared by stirring resorcinol, formaldehyde, sodium carbonate, and deionized (DI) water in a molar ratio of 50:100:1:300. The secondary gel precursor may further use toluenesulfonic acid and calcium carbonate as additives. The use of the additives improves the strength and hardness of a porous structure.

The secondary gel precursor 125 is added to the stack of the secondary sacrificial templates 121, as shown in (f) of FIG. 15, followed by gelation by heating at 50-80° C. for 48-72 hours. Here, the secondary gel precursor 125 is easily added under a reduced pressure of 0.1 atm or less.

The structure including the gelled secondary gel precursor 125 is secondarily carbonized in a nitrogen atmosphere, for example, by heating at 850-1000° C. for 2-3 hours (S470).

By this secondary carbonization, the filler 115 and the secondary sacrificial templates 121 are removed and the gelled secondary gel precursor 105 is carbonized to form a frame made of a carbon material, as shown in (g) of FIG. 15. The frame constitutes a second porous structure 120 surrounding the first porous structure 110.

Then, one surface of the second porous structure 120 is optionally immersed in an aqueous solution, followed by plating to form an electrode 130. The aqueous solution contains a material for the electrode 130. Specifically, only the lower end portion of the second porous structure 120 is immersed, for example, in an aqueous solution of platinum, and is then electroplated by the application of a voltage of 0.1-0.5 V for 100-2000 seconds to plate the electrode 130 with platinum nanoparticles.

According to one embodiment of the present invention, the previous steps S440, S450, S460, and S470 may be repeated to produce a porous hierarchical structure further including a third porous structure having a plurality of third pores whose diameter is smaller than that of the second pores and surrounding the second porous structure 120 and a fourth porous structure having a plurality of fourth pores whose diameter is smaller than that of the third pores and surrounding the third porous structure.

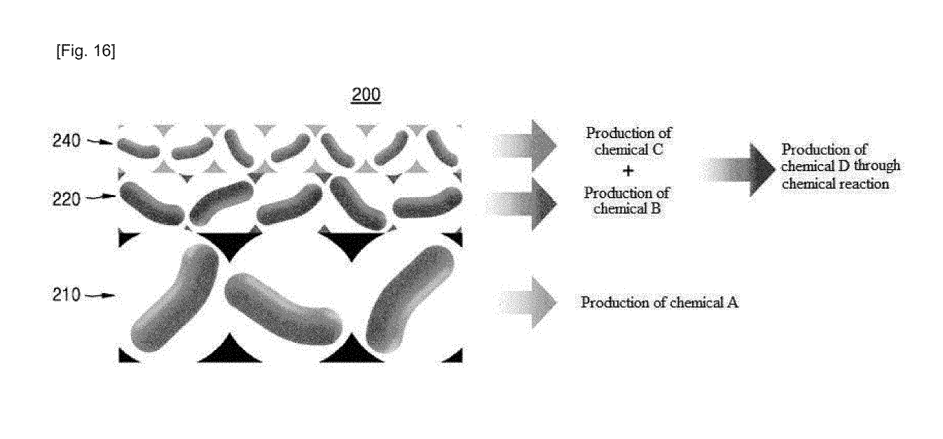

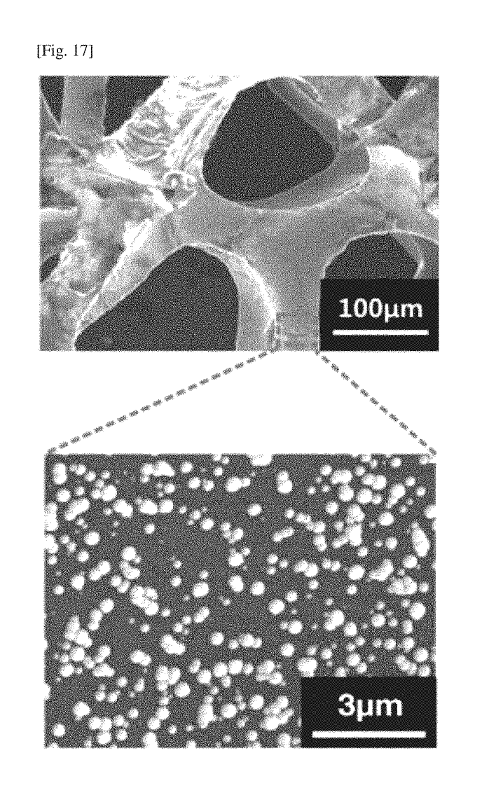

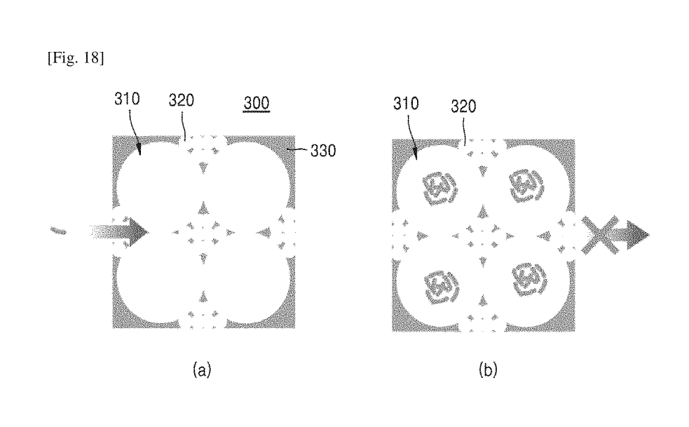

That is, a porous hierarchical structure 200 according to a second embodiment of the present invention can be produced which includes a first porous structure 210 having a plurality of 3-dimensionally interconnected first pores, a second porous structure 220 having a 3-dimensionally interconnected second pores whose diameter is different from that of the first pores and surrounding and bonded to one side of the first porous structure 210, and a third porous structure 240 having a 3-dimensionally interconnected third pores whose diameter is different from that of the second pores and bonded to and surrounding one side of the second porous structure 220, as shown in FIG. 16. It should be understood that the porous hierarchical structure 200 may further include a fourth porous structure (not shown) having a plurality of 3-dimensionally interconnected fourth pores whose diameter is different from that of the third pores and bonded to and surrounding one side of the third porous structure 240.

Thus, the porous hierarchical structure 200 according to the second embodiment of the present invention is defined as a cascade porous hierarchical structure having pores whose sizes are different from area to area so that microbes adapted to the pore sizes can be cultured. Therefore, various chemicals can be produced in one porous hierarchical scaffold. The use of the porous hierarchical structure also enables the production of new chemicals through reactions of chemicals produced from microbes cultured in each pore.

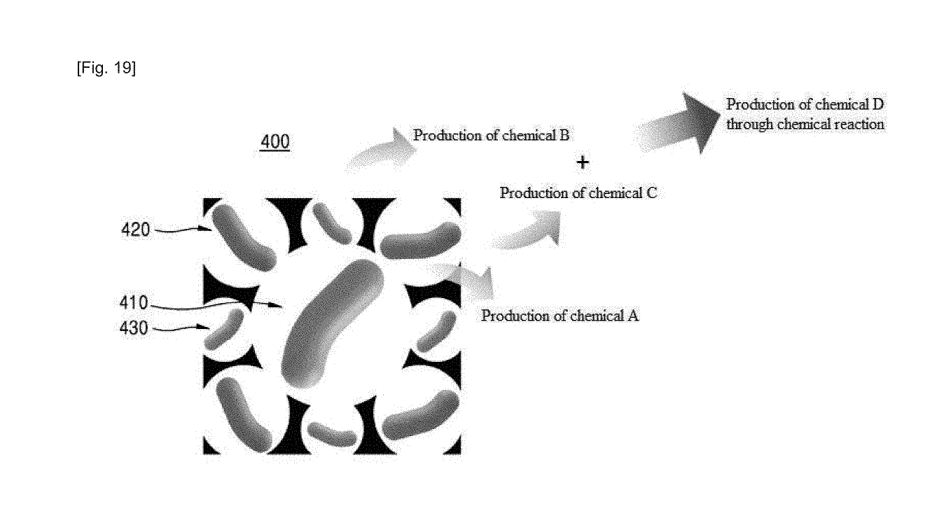

An explanation will be given concerning porous hierarchical structures having smaller diameter pores formed around larger diameter pores with reference to FIGS. 18 and 19. FIG. 18 shows cross-sectional diagrams of a porous hierarchical structure according to a third embodiment of the present invention and FIG. 19 is an exemplary diagram showing a porous hierarchical structure according to a fourth embodiment of the present invention.

The porous hierarchical structure 300 according to the third embodiment of the present invention is constituted by a frame 330 made of a material selected from carbon materials, metal materials, such as nickel (Ni), copper (Cu), and silicon, and metal oxides, such as titanium dioxide (TiO₂) and includes a plurality of 3-dimensionally interconnected first pores 310 having a diameter in the micrometer range and 3-dimensionally interconnected second pores 320 formed around the first pores 310 and whose diameter is smaller than that of the first pores 310, as shown in (a) and (b) of FIG. 18.

Due to this construction, microbes at the initial stage of growth can enter the inner first pores 310 through the outer second pores 320, as shown in (a) of FIG. 18. Thereafter, the microbes proliferate in the first pores, resulting in an increase in population, as shown in (b) of FIG. 18. Accordingly, the grown microbes fail to escape through the second pores, considerably reducing the probability that the microbes will escape from the porous hierarchical structure.

In contrast, the porous hierarchical structure 400 according to the fourth embodiment of the present invention includes a plurality of 3-dimensionally interconnected first pores 410 having a diameter in the micrometer range, 3-dimensionally interconnected second pores 420 formed around the first pores 410 and whose diameter is smaller than that of the first pores 410, and 3-dimensionally interconnected third pores 430 formed around the second pores 420 and whose diameter is smaller than that of the second pores 420, as shown in FIG. 19. It is to be understood that the porous hierarchical structure 400 according to the fourth embodiment of the present invention may further include 3-dimensionally interconnected pores (not shown) formed around the first pores 410 and whose diameter is smaller than that of the third pores 430.

The porous hierarchical structure 400 according to the fourth embodiment of the present invention has pores whose sizes are different from area to area so that microbes of various sizes can be cultured. Therefore, various chemicals can be produced in one porous hierarchical scaffold. The use of the porous hierarchical structure also enables the production of new chemicals through reactions of chemicals produced from microbes cultured in each pore.

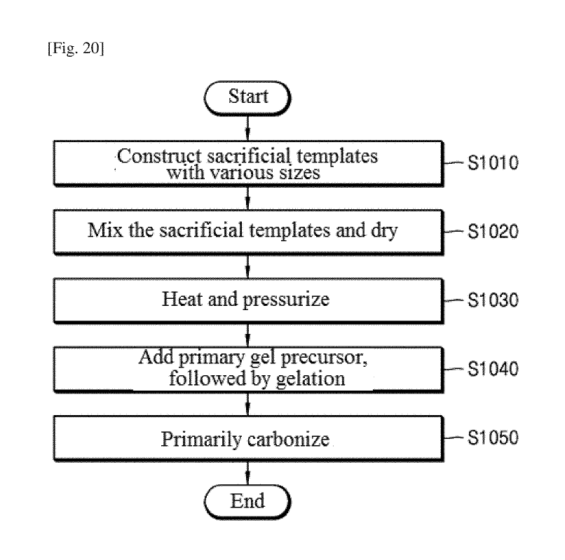

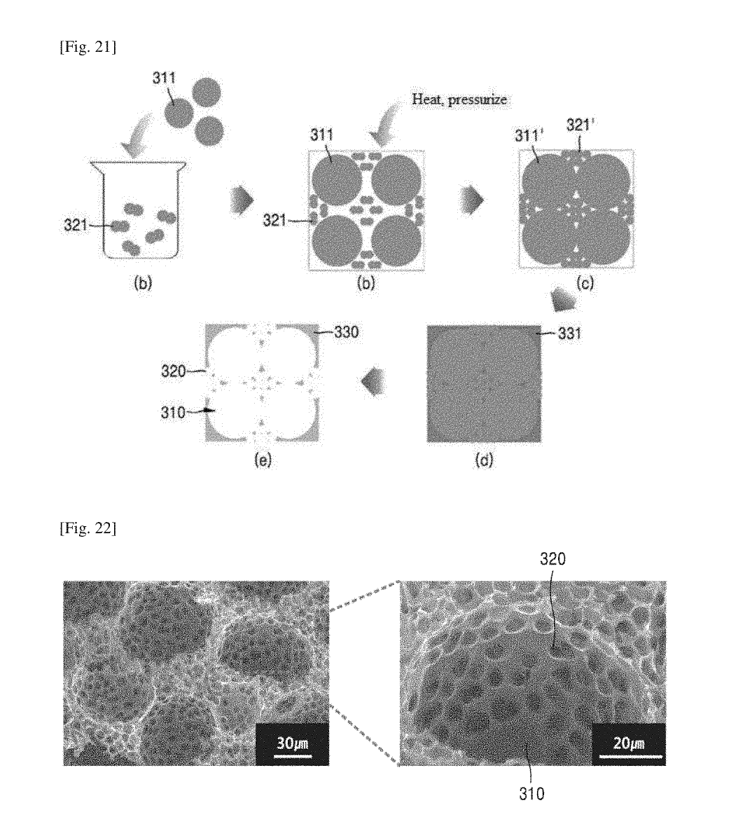

A method for producing the porous hierarchical structure having smaller diameter pores around larger diameter pores according to the third embodiment of the present invention will be explained with reference to FIGS. 20 to 22. FIGS. 20 and 21 are a flow chart and exemplary diagrams explaining the method, respectively, and FIG. 22 shows SEM images of the porous hierarchical structure. Here, the method is explained based on the porous hierarchical structure 300 according to the third embodiment of the present invention but is not limited thereto. For example, the method can be applied to the porous hierarchical structure 400 according to the fourth embodiment of the present invention.

According to the method, the hierarchical porous structure is produced by the following procedure. First, sacrificial templates 311 and 321 having sizes corresponding to pores of various sizes are constructed by a polymerization reaction (S1010), as shown in FIG. 20.

Specifically, the sacrificial templates 311 and 321 are in the form of spheres and can be constructed using a polymer, such as polystyrene (PS), polymethyl methacrylate (PMMA) or polypropylene (PP), or an oxide, such as silicon dioxide (SiO₂) or titanium dioxide (TiO₂), as shown in (a) of FIG. 21.

The plurality of sacrificial templates 311 and 321 are primarily stirred in a solution of sodium dodecylsulfate as a surfactant and cyclohexane as a swelling agent.

Subsequently, a cross-linking agent (e.g., divinylbenzene), a polymerization initiator (e.g., benzoyl peroxide), and optionally a monomer (e.g., styrene) are secondarily stirred in the primary stirred solution.

The secondarily stirred solution is mixed with a polyvinyl alcohol solution and is polymerized by heating at 70-80° C. for 12-18 hours.

After completion of the polymerization, the above procedure from the primary stirring to the polymerization is repeated once for the sacrificial templates 311 and 321 to construct double spherical templates, like the secondary sacrificial template 321 shown in (a) of FIG. 21.

Particularly, the sizes of the plurality of sacrificial templates 311 and 321 can be increased by varying the amount of the optional monomer during the secondary stirring so that the sizes of pores can be determined.

Then, the sacrificial templates are mixed in a predetermined mass ratio in an ethanolic solution, followed by drying (S1020).

Specifically, the sacrificial templates 311 and 321 are mixed in a predetermined mass ratio. For example, the secondary sacrificial templates 321 having a relatively small size and the primary sacrificial templates 311 are mixed in a mass ratio of 5:1 to 10:1. It should be understood that a plurality of other sacrificial templates having different diameters can be further mixed with the primary sacrificial templates 311 and the secondary sacrificial templates 321 in a predetermined mass ratio.

Thereafter, the ethanolic solution is evaporated by natural drying, leaving a stack in which the primary sacrificial templates 311 and the secondary sacrificial templates 321 are mixedly present.

The mixed stack of the primary sacrificial templates 311 and the secondary sacrificial templates 321 is pressurized to 10-500 kPa and heated to 110-150° C. (S1030). As a result, the contact area between the primary sacrificial templates 311 and the secondary sacrificial templates 321 is enhanced, as shown in (c) of FIG. 21.

The contact area between the primary sacrificial templates 311 and the secondary sacrificial templates 321 is determined by 3-dimensional interconnection structures between first pores 310 and second pores 320 to be formed. Thus, the pressure and temperature conditions can be determined depending on the material for the primary sacrificial templates 311 and the secondary sacrificial templates 321 and the size of the contact area.

A primary gel precursor 331 is added to the pressurized and heated stack of the primary sacrificial templates 311′ and the secondary sacrificial templates 321′, followed by gelation by heating (S1040).

Here, the primary gel precursor 331 may be prepared by stirring resorcinol, formaldehyde, sodium carbonate, and deionized (DI) water in a molar ratio of 50:100:1:300. The gel precursor may further use toluenesulfonic acid and calcium carbonate as additives. The use of the additives improves the strength and hardness of a porous structure.

The primary gel precursor 331 is added to the pressurized and heated mixed stack of the primary sacrificial templates 311′ and the secondary sacrificial templates 321′, followed by gelation by heating at 50-80° C. for 48-72 hours, as shown in (d) of FIG. 21. Here, the primary gel precursor 331 is easily added under a reduced pressure of 0.1 atm or less.

The mixed stack including the gelled primary gel precursor 331 is primarily carbonized in a nitrogen atmosphere, for example, by heating at 800-1000° C. for 2-3 hours (S1050).

By this primary carbonization, the primary sacrificial templates 311′ and the secondary sacrificial templates 321′ are removed and the gelled primary gel precursor 331 is carbonized to form a frame 330, as shown in (e) of FIG. 21 and FIG. 22. The frame 330 constitutes the hierarchical structure 300 including a plurality of 3-dimensionally interconnected first pores 310 having a diameter in the micrometer range and a plurality of 3-dimensionally interconnected second pores 320 formed around the first pores 310 and whose diameter is smaller than that of the first pores 310.

Thereafter, steps S1010 to S1050 are repeated to produce a porous hierarchical structure further including porous structures having different pores formed on the outer surface of the porous hierarchical structure 300.

The method according to the embodiment of the present invention facilitates the production of the porous hierarchical structure having smaller diameter pores around larger diameter pores. Due to this construction, the porous hierarchical structure is suitable for preventing microbes proliferated in the pores from escaping and culturing microbes of various sizes in the pores.

The scope and spirit of the present invention have been described with reference to the foregoing preferred embodiments. However, it is to be noted that these embodiments are provided for illustrative purposes and are not intended to limit the scope and spirit of the present invention.

Those skilled in the art will appreciate that modifications can be made to these embodiments without departing from the scope and spirit of the present invention.

### INDUSTRIAL APPLICABILITY

The porous structure of the present invention is constituted by a frame and has a plurality of pores distributed in a closest packed state. The porosity of the porous structure according to the present invention can be maximized because the plurality of pores defined by the frame are distributed in a closest packed state and are interconnected 3-dimensionally through a plurality of connecting passages in a symmetric structure. Therefore, the porous structure of the present invention is useful in industrial applications.

In addition, the porous hierarchical structure of the present invention is effective in preventing microbes from escaping and is thus industrially useful.

1. A porous structure constituted by a frame having a plurality of pores interconnected 3-dimensionally through a plurality of connecting passages.

2. The porous structure according to claim 1, wherein the frame is made of a material selected from carbon materials, metal materials, and metal oxides.

3. The porous structure according to claim 1, wherein the pores have a diameter in the micrometer range.

4. The porous structure according to claim 1, wherein four connecting passages extend downwardly, four connecting passages extend laterally, and four connecting passages extend upwardly from the central pore.

5. A method for producing a porous structure comprising (A) constructing and stacking a plurality of sacrificial templates, (B) pressurizing and heating the stack of the sacrificial templates, (C) adding a gel precursor to the pressurized and heated stack of the sacrificial templates, followed by gelation, and (D) carbonizing the stack of the sacrificial templates comprising the gelled gel precursor.

6. The method according to claim 5, wherein step (A) comprises (A-1) preparing a plurality of sacrificial templates in the form of spheres by a polymerization reaction using a polymer or an oxide and (A-2) drying the plurality of sacrificial templates during slow cooling or freezing to stack the plurality of sacrificial templates.

7. The method according to claim 6, wherein, in sub-step (A-2), the stack comprises a hexagonal closest packed structure and a cubic closest packed structure.

8. A porous hierarchical structure comprising a first porous structure having a plurality of 3-dimensionally interconnected first pores and a second porous structure having a plurality of 3-dimensionally interconnected second pores whose diameter is different from that of the first pores and surrounding and bonded to the first porous structure.

9. The porous hierarchical structure according to claim 8, further comprising an electrode disposed on one outer surface of the second porous structure.

10. The porous hierarchical structure according to claim 8, wherein each of the first porous structure and the second porous structure is made of a material selected from carbon materials, metal materials, and metal oxides.

11. The porous hierarchical structure according to claim 8, wherein the first pores have a diameter in the micrometer range and the second pores have a diameter smaller than that of the first pores.

12. The porous hierarchical structure according to claim 8, further comprising a third porous structure having a plurality of third pores whose diameter is smaller than that of the second pores and surrounding the second porous structure and a fourth porous structure having a plurality of fourth pores whose diameter is smaller than that of the third pores and surrounding the third porous structure.

13. A method for producing a porous hierarchical structure comprising (A) constructing a first porous structure having a plurality of 3-dimensionally interconnected first pores and (B) constructing a second porous structure having a plurality of 3-dimensionally interconnected second pores whose diameter is different from that of the first pores and surrounding and bonded to the first porous structure.

14. The method according to claim 13, further comprising (C) constructing a third porous structure having a plurality of third pores whose diameter is smaller than that of the second pores and surrounding the second porous structure and (D) constructing a fourth porous structure having a plurality of fourth pores whose diameter is smaller than that of the third pores and surrounding the third porous structure.

15. A porous hierarchical structure comprising a frame having a plurality of 3-dimensionally interconnected first pores having a diameter in the micrometer range and a plurality of 3-dimensionally interconnected second pores formed around the first pores and whose diameter is smaller than that of the first pores.

16. The porous hierarchical structure according to claim 15, wherein the frame is made of a material selected from carbon materials, metal materials, and metal oxides.

17. The porous hierarchical structure according to claim 15, further comprising a plurality of 3-dimensionally interconnected third pores formed around the first pores and whose diameter is smaller than that of the second pores.

18. The porous hierarchical structure according to claim 17, further comprising a plurality of 3-dimensionally interconnected fourth pores formed around the first pores and whose diameter is smaller than that of the third pores.

19. A method for producing a porous hierarchical structure comprising (A) constructing sacrificial templates having different sizes corresponding to micrometer- to nanometer-sized pores by a polymerization reaction, (B) mixing the sacrificial templates in a predetermined mass ratio and drying the sacrificial templates to stack the sacrificial templates, (C) pressurizing and heating the mixed sacrificial template stack, (D) adding a primary gel precursor to the mixed sacrificial template stack, followed by gelation, and (E) primarily carbonizing the mixed sacrificial template stack comprising the gelled primary gel precursor.

20. The method according to claim 19, wherein, in step (A), preparing sacrificial templates in the form of spheres using a polymer or an oxide.
